(19) United States
(12) Patent Application Publication
(10) Pub. No.: US 2025/0312061 A1
(43) Pub. Date: Oct. 9, 2025

(54) Arthroscopic Cannula and Insertion Tool

(71) Applicant: ConMed Corporation, Largo, FL (US)

(72) Inventors: Carlton Owens, Jacksonville, FL (US); Eric Romero, JR., Ridge Manor, FL (US); Benjamin Barrett, Parrish, FL (US)

(73) Assignee: ConMed Corporation, Largo, FL (US)

(21) Appl. No.: 19/170,354

(22) Filed: Apr. 4, 2025

Related U.S. Application Data
(60) Provisional application No. 63/631,046, filed on Apr. 8, 2024.

(51) Int. Cl. A61B 17/34

(52) U.S. Cl. CPC A61B 17/3423

(57) ABSTRACT

A uniquely structured cannula and an instrument to efficiently and effectively deploy and remove the cannula is provided. In particular, the present disclosure is directed to inventive cannula device embodiments structured and/or configured to have a working portal (for delivery of biologics or use by other surgical devices), and a method to easily deploy and remove the cannula while reducing the area of the surgical site required for insertion therein.

### CROSS-REFERENCE TO RELATED APPLICATION

The present application claims priority to and the benefit of U.S. Provisional Patent Application No. 63/631,046 filed on Apr. 8, 2024.

### BACKGROUND

### 1. Field

The present disclosure relates to a surgical device and, more particularly, to a cannula with an insertion tool.

### 2. Description of the Related Art

Cannulas are used to support arthroscopic and endoscopic surgical procedures by providing access through portals to a surgical site. The access portals can be used, for example, by a variety of surgical devices and instruments including cameras, electrosurgical probes, bone anchor delivery devices, and biologic delivery devices.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY

The inventors recognize that during arthroscopic delivery of surgical devices to a surgical site, it can be difficult to insert and remove the cannula from an opening in the body where the cannula provides access to a surgical site. The inventors further recognize that conventional cannulas often require a large opening through the body and/or surgical site to insert the cannula therethrough/therein.

It is therefore a principal object and advantage of embodiments of the present disclosure to provide a uniquely structured cannula and an instrument to efficiently and effectively deploy and remove the cannula, and that eliminates one or more of the problems/issues/deficiencies associated with conventional cannulas. In particular, the present disclosure is directed to inventive cannula device embodiments structured and/or configured to have a working portal (for delivery of biologics or use by other surgical devices), and a method to easily deploy and remove the cannula while providing the ability to reduce the area of the surgical site required for insertion therein.

According to an embodiment, a cannula is provided having a first flange positioned at a first end, a second flange positioned at a second end, a cannulated main body including a working portal extending between the first and second ends, and a feeding mechanism or insertion/deployment tool to deploy and remove the cannula through/to/from an opening in body tissue and the surgical site. The first flange of the cannula can have a pair of wings that are capable of folding in toward each other by pinching the first flange with the feeding mechanism, thereby decreasing the effective length of the first flange that is perpendicular to the cannula's central longitudinal axis and thus the area required for the site of insertion. An intermediate flange can also be included and positioned on the cannulated main body, and also be movable about the cannulated main body (e.g., rotationally, axially).

According to an embodiment, a cannula can include at least one seal, spacers between seals, and/or flexible threads, and be made of material(s) as discussed and described (see, e.g., U.S. patents application Ser. Nos. 20/210,267631, 20220054164, and 20210177459). The material can be translucent, opaque, and/or colors that can assist with radiology (as should be understood by a person of ordinary skill in the art in conjunction with a review of this disclosure).

These and other aspects of the embodiments described herein will be apparent from and elucidated with reference to the embodiment(s) described hereinafter.

### BRIEF DESCRIPTION OF THE SEVERAL VIEWS OF THE DRAWING(S)

The present embodiments will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1 is a distal front or side perspective view of a cannula, according to an embodiment.

FIG. 2 is a proximal front or side perspective view of the cannula, according to an embodiment.

FIG. 3 is an exploded perspective view of the cannula, according to an embodiment.

FIG. 4 is another top perspective view of the cannula with the intermediate flange removed for illustration purposes, according to an embodiment.

FIG. 5 is a close-up perspective view of the first end of the cannula, according to an embodiment.

FIG. 6 is a front or side perspective view of a feeding mechanism, according to an embodiment.

FIG. 7 is a rear or other side perspective view of the feeding mechanism, according to an embodiment.

FIG. 8 is a perspective view of the feeding mechanism inserted into the cannula and preparing to insert the cannula into a surface in a subsequent step, according to an embodiment.

FIG. 9 is a close-up view of the feeding mechanism inserting the cannula into a surface, according to an embodiment.

FIG. 10 is a perspective view of the cannula inserted into a surface (e.g., skin), according to an embodiment.

FIG. 11 is a perspective view of the cannula inserted into a surface with the intermediate flange positioned adjacent to the surface, according to an embodiment.

FIG. 12 is a close-up view of the first flange inserted through tissue and past the inner surface of the tissue (e.g., skin), according to an embodiment.

FIG. 13 is a perspective view of a user rotating the intermediate flange and moving the same axially to remove the cannula from a surface in a subsequent step, according to an embodiment.

FIG. 14 is a close-up view of a user removing the cannula from a surface using the feeding mechanism, according to an embodiment.

FIG. 15 is a close-up view of the feeding mechanism positioning the wings in the engaged position underneath a surface (or adjacent to the inner surface of tissue), according to an embodiment.

FIG. 16 is a perspective view of a user using the feeding mechanism to remove the cannula from a surface in a subsequent step, according to an embodiment.

FIG. 17 is a perspective view of the cannula removed from a surface using the feeding mechanism, according to an embodiment.

### DETAILED DESCRIPTION

Aspects of the present disclosure and certain features, advantages, and details thereof, are explained more fully below with reference to the non-limiting examples illustrated in the accompanying drawings. Descriptions of well-known structures are omitted so as not to unnecessarily obscure the described embodiments of the disclosure in detail. It should be understood, however, that the detailed description and the specific non-limiting examples, while indicating aspects of the embodiments, are given by way of illustration only, and are not by way of limitation. Various substitutions, modifications, additions, and/or arrangements, within the spirit and/or scope of the underlying inventive concepts will be apparent to those skilled in the art from this disclosure.

While embodiments of the present disclosure have been particularly shown and described with reference to certain exemplary embodiments, it will be understood by one skilled in the art that various changes in detail may be affected therein without departing from the spirit and scope of the disclosure as defined by claims that can be supported by the written description and drawings. Further, where exemplary embodiments are described with reference to a certain number of elements it will be understood that the exemplary embodiments can be practiced utilizing either less than or more than the certain number of elements. If elements shown in a particular Figure discussed below are not specifically identified with respect to that Figure, the elements should be sufficiently identified with respect to at least one other Figure.

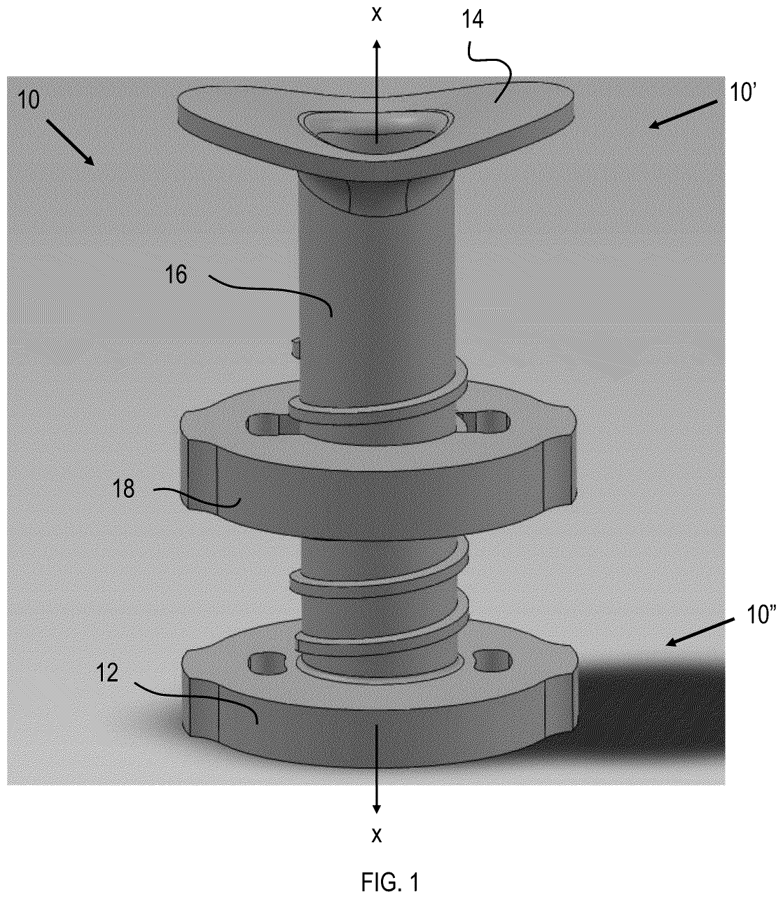

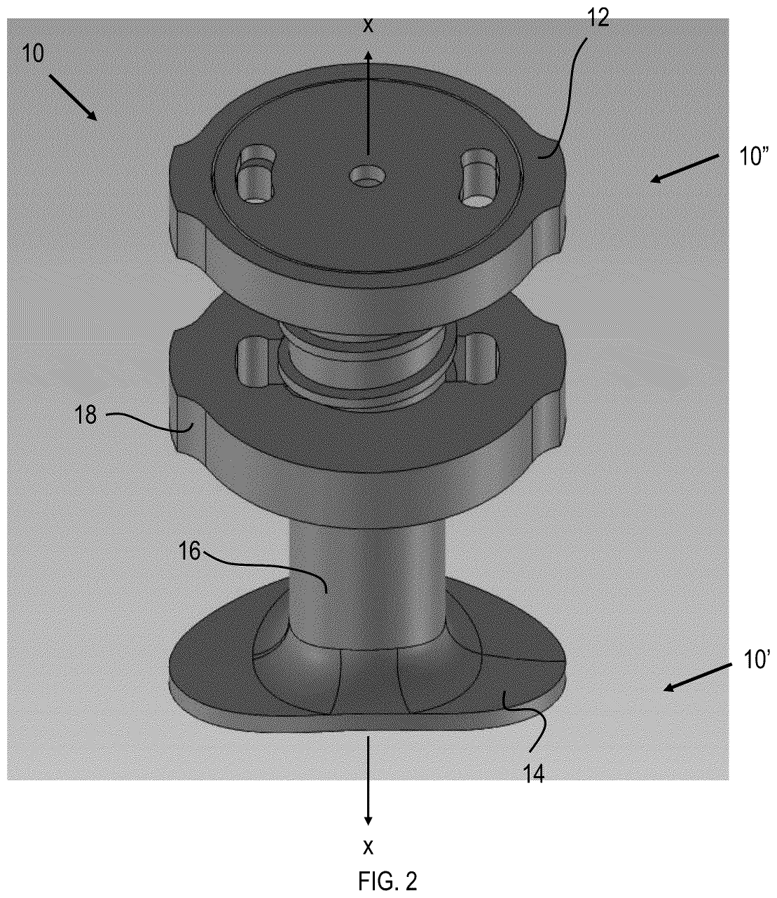

Referring now to the figures, wherein like reference numerals refer to like parts throughout, FIGS. 1 and 2 show distal/front and proximal/front view schematic representations, respectively, of a cannula 10 according to an embodiment. The cannula 10 can include a first flange 14 positioned at a first/distal end 10′, a second flange 12 positioned at a second/proximal end 10″, and a cannulated body 16 extending along a central longitudinal axis x-x therebetween forming a portal therethrough. In some embodiments, a third or intermediate flange 18 can be positioned about the cannulated body 16 between the first flange 14 and second flange 12 (and can be rotated about and/or move axially along the cannulated body 16, and can have inner threads that mate with outer threads of the cannulated body 16).

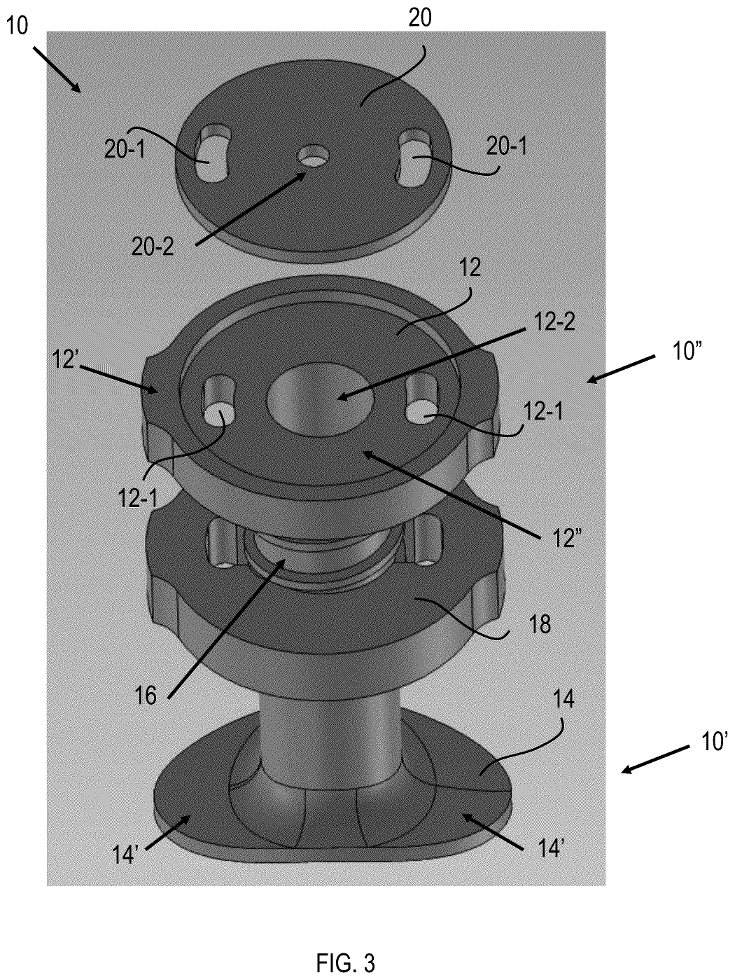

Referring now to FIG. 3, an exploded view of the cannula 10 is shown. The cannula 10 can have an external seal 20 positioned over the second flange 12. The external seal 20 can have a first pair of apertures 20-1 and a second aperture 20-2 that align with apertures of the second flange 12 and apertures with the intermediate flange 18, and also with the wings of the first flange 14′, as described in further detail below.

As shown in FIG. 3, the second flange 12 is positioned at the second end of the cannula 10″ and can further comprise an outer surface 12′ and an inner surface 12″. The outer surface 12′ can have a diameter that largely aligns with the diameters of the outer surfaces of the intermediate flange 18 and first flange 14. In accordance with other embodiments, the diameter of the first flange 14 can be the same as or smaller than the diameter of the second flange 12 and intermediate flange 18 (and the first flange 14 can be an elliptical, oval, or otherwise elongated shape). The inner surface 12″ of the second flange 12 can further comprise a pair of first apertures 12-1 and a central aperture 12-2. In some embodiments, the first apertures 12-1 can be elongated and dimensioned to accept a feeding mechanism, as described below, and the central aperture 12-2 can be in communication with the cavity of the cannulated body 16. In some embodiments, the inner surface 12″ can be recessed with respect to the outer surface 12′ such that the external seal 20 can be positioned within the recess and on the inner surface 12″ and be either under or substantially flush with the outer surface 12′ as shown in FIG. 2. While a pair of apertures is discussed with respect to the second flange 12 and external seal 20, it is to be understood that any number or combination of apertures could be formed therethrough (including as shown and lined up with apertures of the flanges).

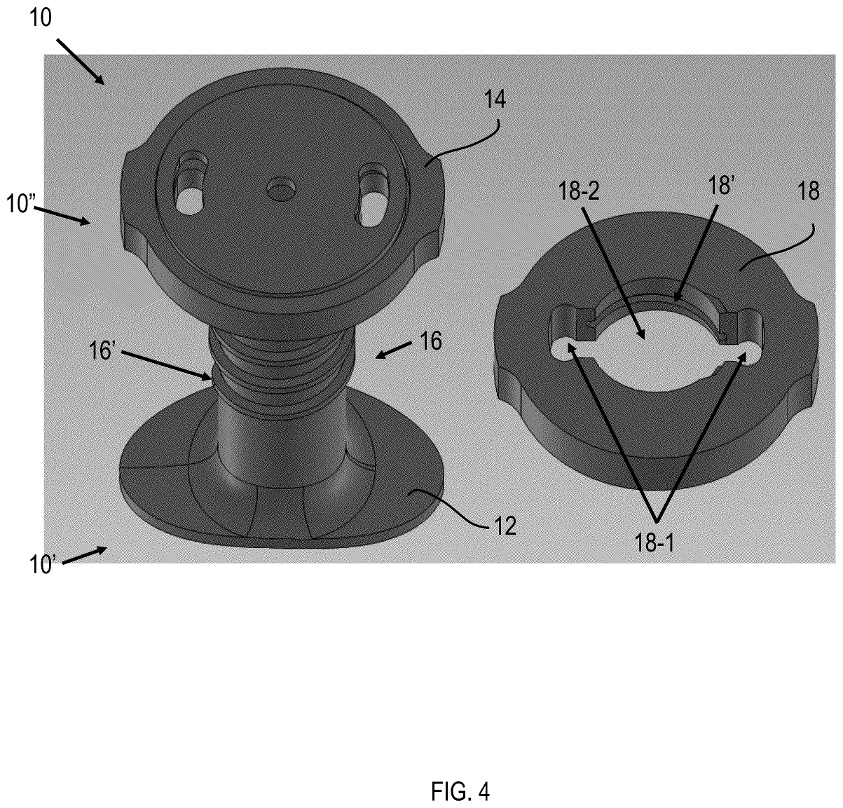

Referring now to FIG. 4, the cannula 10 with the intermediate flange 18 removed for illustration purposes is shown. Intermediate flange 18 can have elongated apertures 18-1 and a circular aperture 18-2. In some embodiments, the elongated apertures 18-1 are in communication with the circular aperture 18-2, as seen in FIG. 4, but can advantageously be of different widths and dimensions such that an insertion device can be placed through the elongated apertures 18-1 without obstructing the cannulated body 16 positioned through the circular aperture 18-2, as explained in further detail below. In this example, the elongated apertures 18-1 align with the first apertures of both the second flange 12-1 and external seal 20-1 and are dimensioned to accept a feeding mechanism/insertion device, as explained below. The circular aperture 18-2 can have a diameter larger than that of the cannulated body 16, allowing the intermediate flange 18 to fit over the cannulated body 16 (and over first flange 12, which can be flexible to allow such fitting over and positioning through aperture 18-2 and which can also include openings from aperture 18-2 into apertures 18-1). In some embodiments, the intermediate flange 18 has a set of threads 18′ on the interior surface thereof that correspond to receiving threads 16′ on the cannulated body 16, as shown in FIG. 4. This allows the intermediate flange 18 to be rotatably coupled to the cannulated body 16 and allow a user to adjust the distance between the intermediate flange 18 and the first flange 14/second flange 12 along the cannulated body 16 by rotating the intermediate flange 18 either clockwise or counterclockwise up or down the cannulated body 16. While elongated and circular apertures are discussed above, it is to be understood that apertures of differing shapes and sizes can be used. Further, threading does not need to be present—the intermediate flange 18 can be friction fit on the cannulated body and moved/slid axially along the cannulated body 16 and axis x-x.

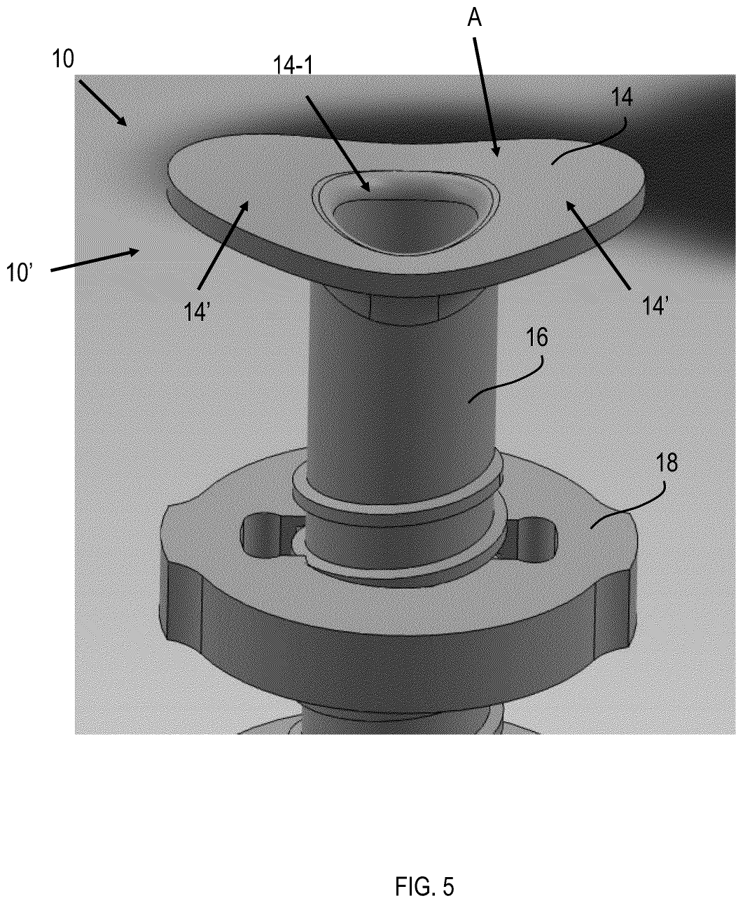

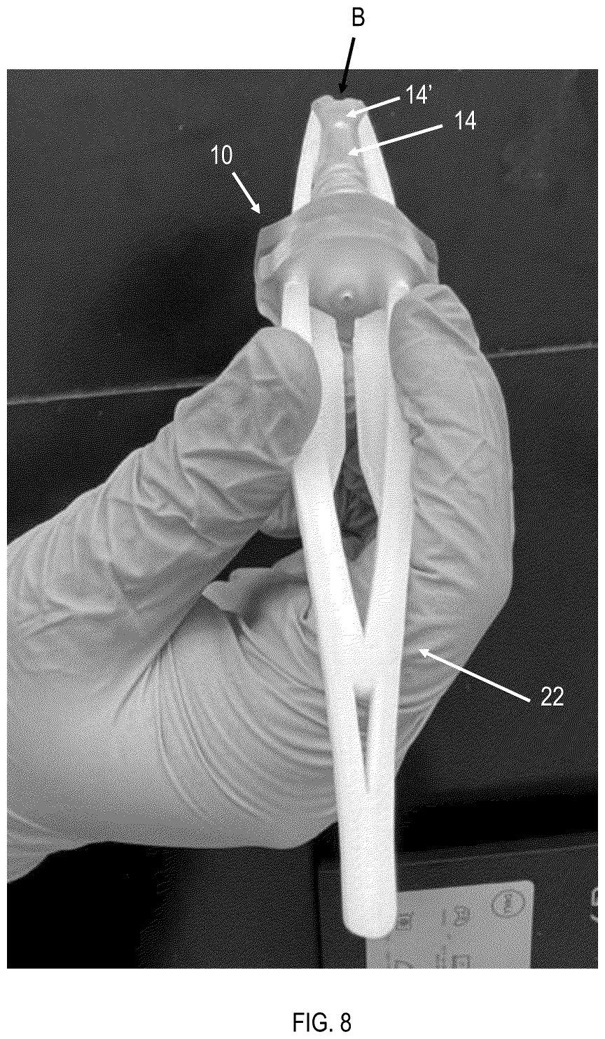

Referring now to FIG. 5, a close-up view of the first flange 14 is shown. The first flange 14 can be coupled to the first end 10′ of the cannula 10 such that an aperture 14-1 of the first flange 14 is in communication with the cavity of the cannulated body 16. First flange 14 can further comprise a pair of wings 14′ that are preferably formed of a flexible material that is configured to flex when a force is applied to the wings 14′ by the insertion devices and/or when the intermediate flange is place on/off the cannulated body (as discussed above, and/or as should be understood by a person of ordinary skill in the art in conjunction with a review of this disclosure). This advantageously allows the wings 14′ to pinch in towards each other from a neutral position A (FIG. 5) to an engaged position B (see FIG. 8), thereby reducing the effective length as measured at an angle to/substantially perpendicular or perpendicular to axis x-x of the first flange 14.

This reduction in the effective length allows the first flange 14 to be inserted into and through a surface and tissue having an opening that is larger than the wings 14′ while in the engaged position B but smaller than the wings 14′ while in the neutral position A (i.e., reducing the area of insertion). In this example, the wings 14′ are in communication with each other, however, in accordance with other embodiments, the wings 14′ can be comprised of separate pieces. In some embodiments, as shown for example in FIG. 1, the wings 14′ are angled distally and toward the aperture 14-1 of the first flange 14 (i.e., slightly concave with respect to the cannulated body 16) and have a length that largely aligns with the length of the intermediate flange 18 and first flange 14 when the wings 14′ are in the neutral position A. In some examples, the length of the first flange 14 is smaller or larger than that of the length of the intermediate flange 18 and the first flange 14. In one preferred embodiment, the length of the first flange 14 is at least of a size wherein the apertures of the second flange 12-1 and intermediate flange 18-1 align with the wing portions 14′, as described in further detail below. In some embodiments, the first flange 14 further comprises one or more indents (not shown) to help align the positioning of the feeding mechanism onto the first flange 14, as described below.

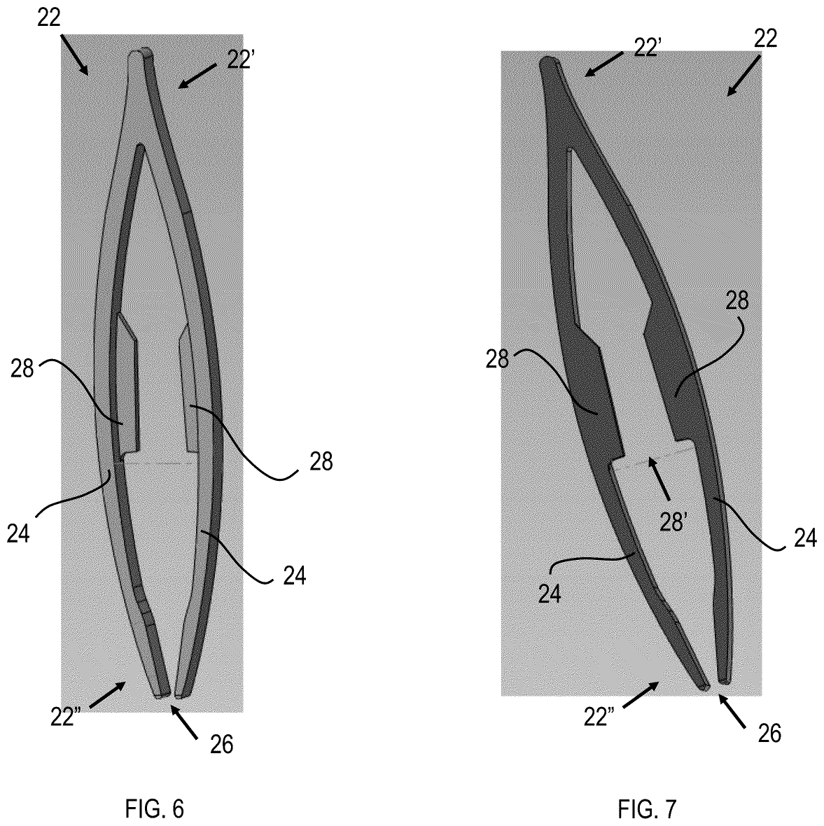

Referring now to FIGS. 6 and 7, a feeding mechanism/insertion device 22 is shown. Feeding mechanism 22 can have a pair of flexible arms 24 that are oblong (or “wishbone”) in shape and connected at a first end 22′. In a preferred embodiment, each arm 24 can have a diameter smaller than that of the apertures of the external seal 20 and second flange 12-1, and elongated apertures of the intermediate flange 18-1, such that the arms 24 of the feeding mechanism 22 can pass through the apertures/elongated portions of the cannula 10 discussed above. In some embodiments, a second end 22″ of each arm 24 comes to a tip 26 that is capable of coming together when pressure is applied to the arms 24 toward each other (neutral position, shown in FIGS. 6-7, can be squeezed together, and then can be biased back to the neutral position). In some embodiments, the tips 26 of the arms 24 are blunted, while in other embodiments, the tips 26 can be rounded or pointed/sharp. In some examples, as shown in FIGS. 6 and 7, a stopper 28 can be positioned on each arm 24 of the feeding mechanism 22 to provide a structure against which the second flange 12 (and/or external seal 20) can rest, allowing a user to know where to position the cannula 10 on the feeding mechanism 22 prior to insertion (i.e., the length of the feeding mechanism 22 from the bottom of the stopper 28′ to the second end 22″ is approximately of the same length as the cannula device 10).

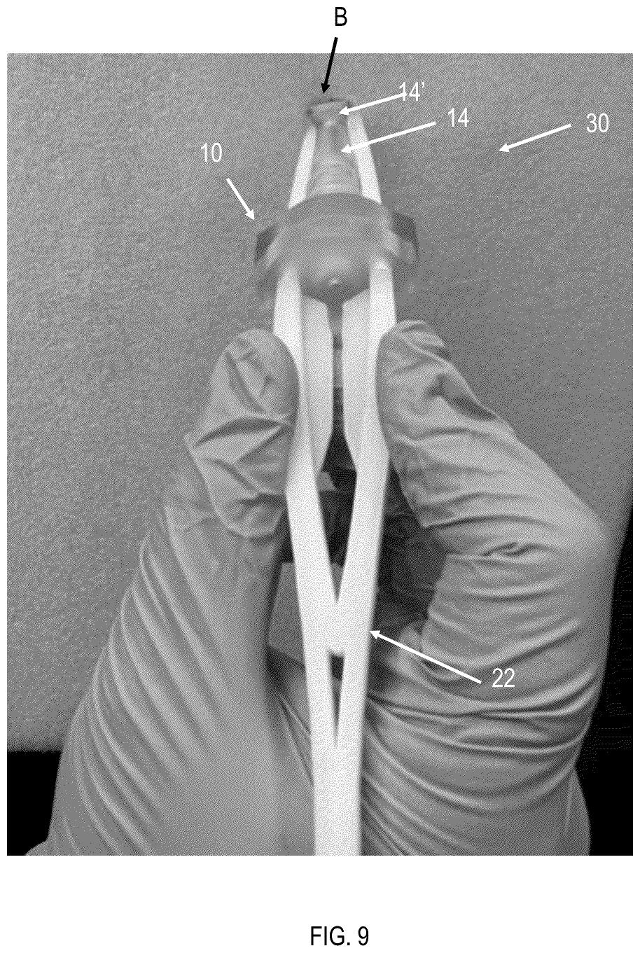

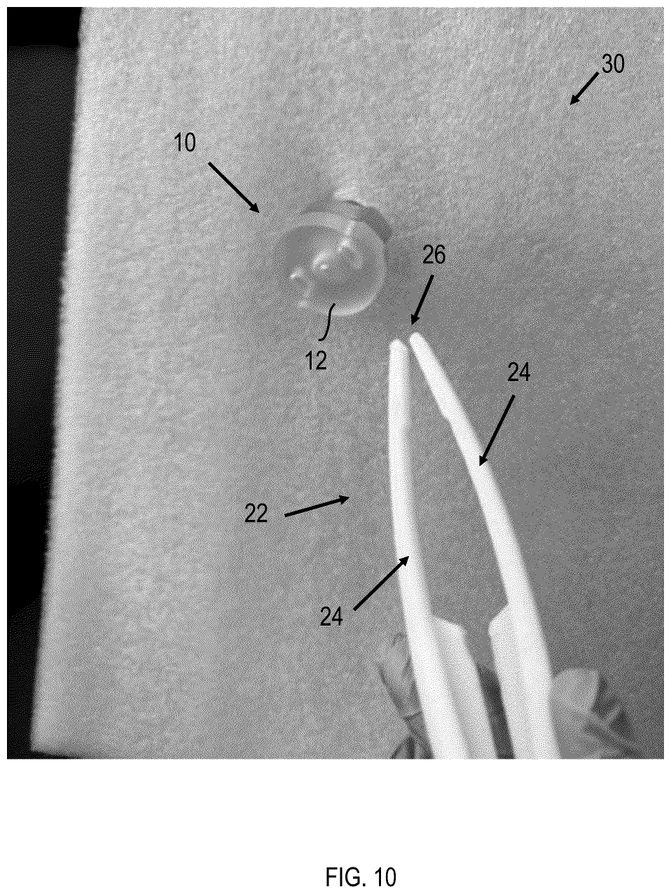

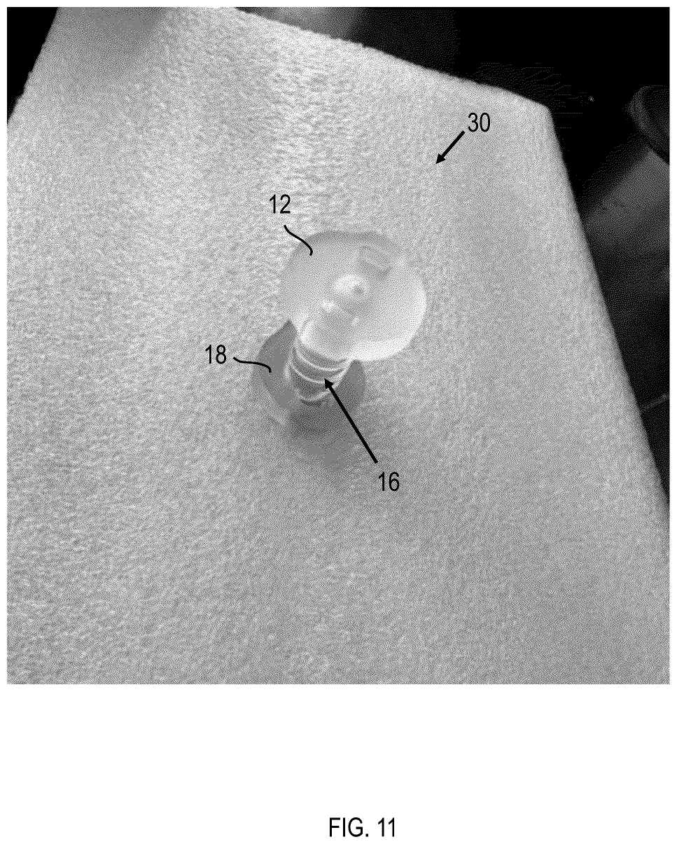

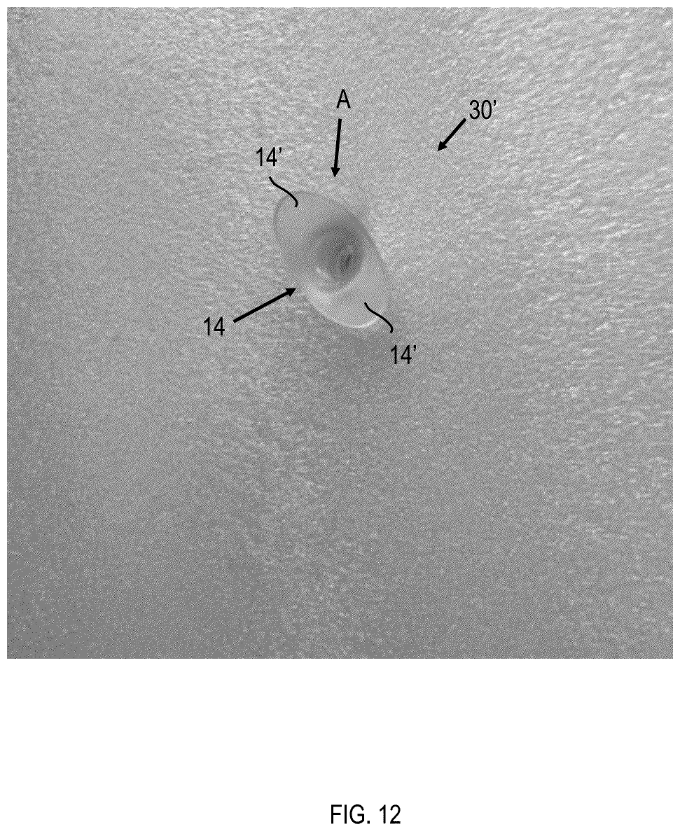

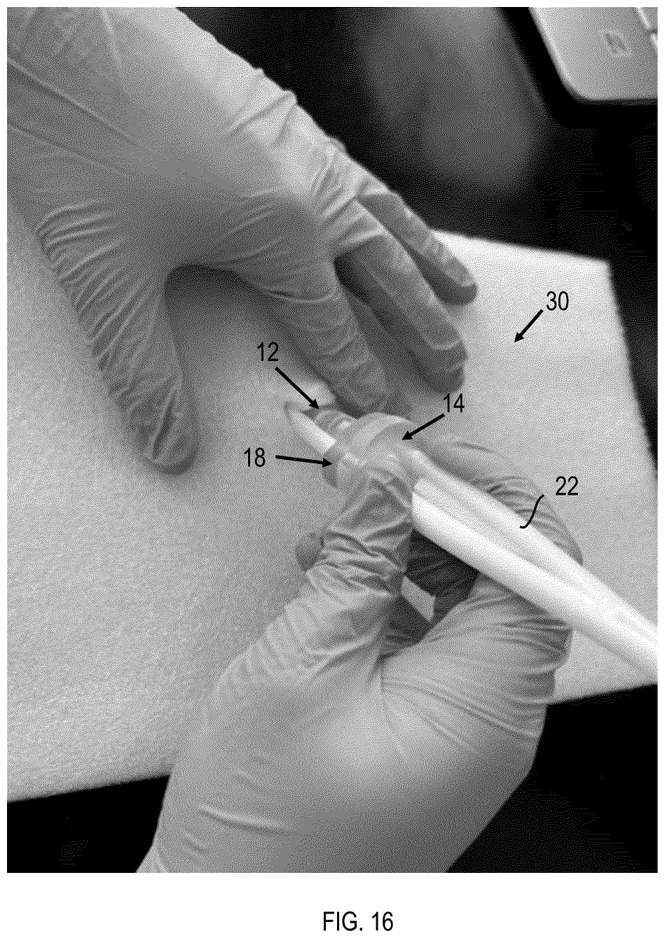

Referring to FIGS. 9-17, the cannula 10 being inserted into, through, and then removed from a substrate or tissue with an outer surface (FIG. 11, 30) and inner surface (FIG. 12, 31) is shown (for the purpose of providing surgical devices and instruments including cameras, electrosurgical probes, bone anchor delivery devices, and biologic delivery devices access to a surgical or other body site that needs treatment or surgery). In a first step, a user can position the cannula 10 on the feeding mechanism 22 by inserting the arms 24 of the feeding mechanism 22 through the second flange first apertures 12-1 and intermediate flange elongated apertures 18-1 and use the tips 26 of the arms 24 to pinch or squeeze the wings 14′ of the first flange 14 to move the wings 14′ to the engaged position B. Next, while still pinching the wings 14′, a user can guide the first flange 14 into an opening in the surface 30. Once the first flange 14 is fully inserted into and through the outer surface 30, the tissue, and the inner surface 30′, a user can release the pinching/squeezing force applied to the feeding mechanism 22, thereby releasing the wings 14′ to the neutral position A underneath the inner surface 30′. The feeding mechanism 22 can be removed from the cannula 10 by guiding the arms 24 through the second and intermediate flanges 12 and 18 while the cannula 10 is held in place underneath the inner surface 30′ by the first flange 14 (FIG. 12).

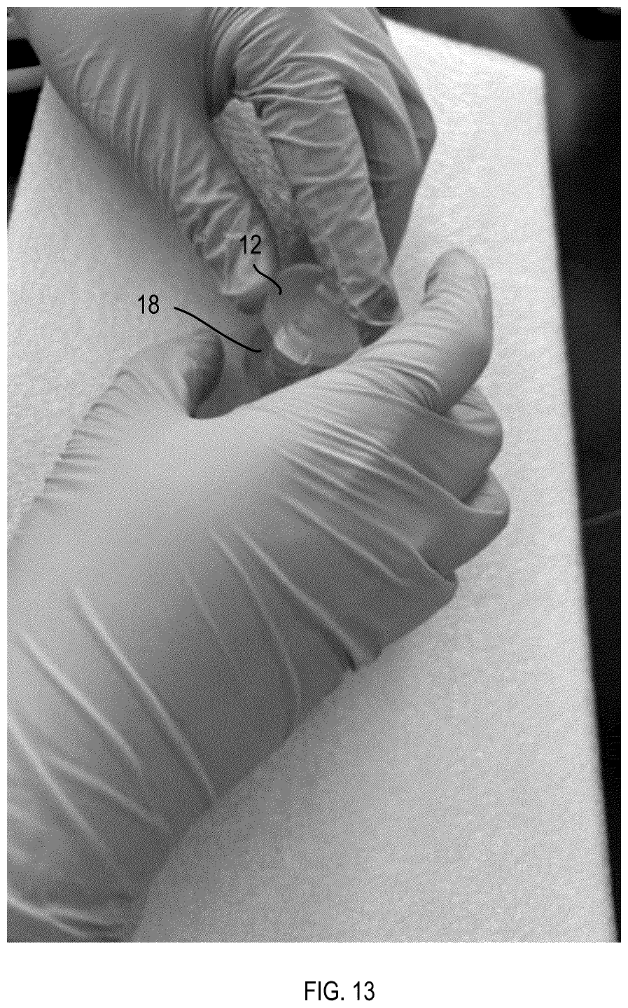

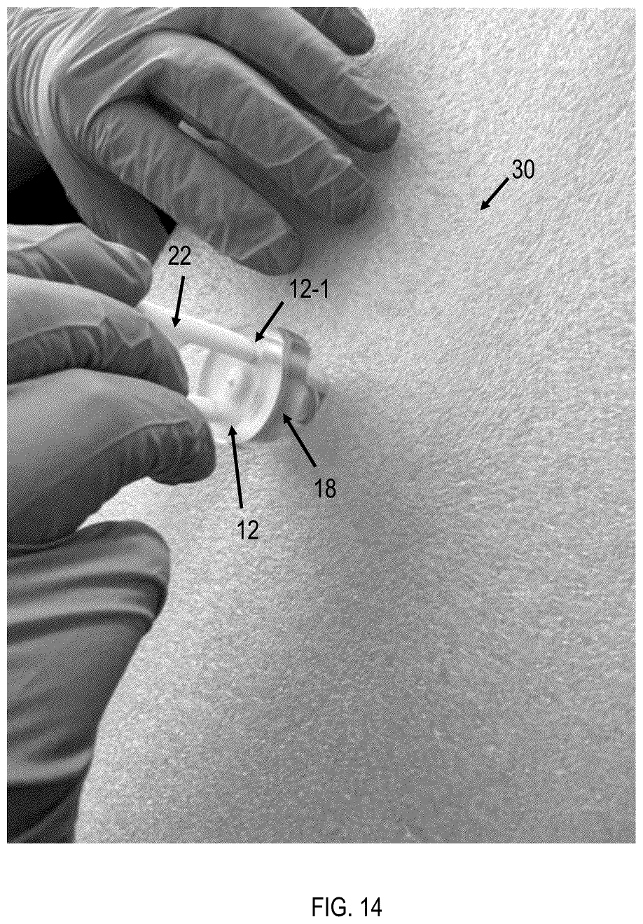

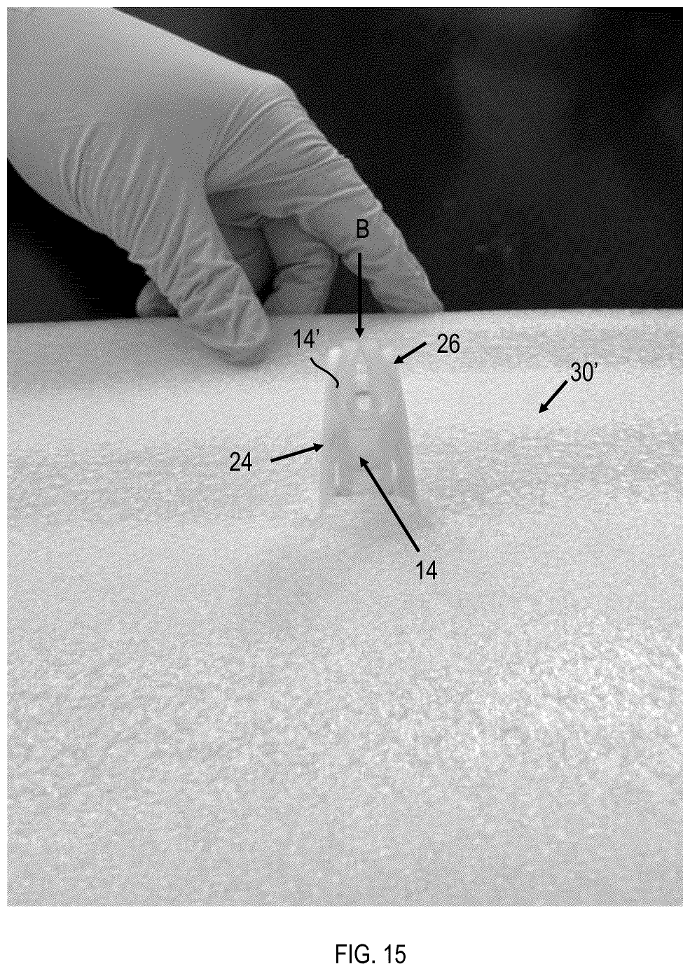

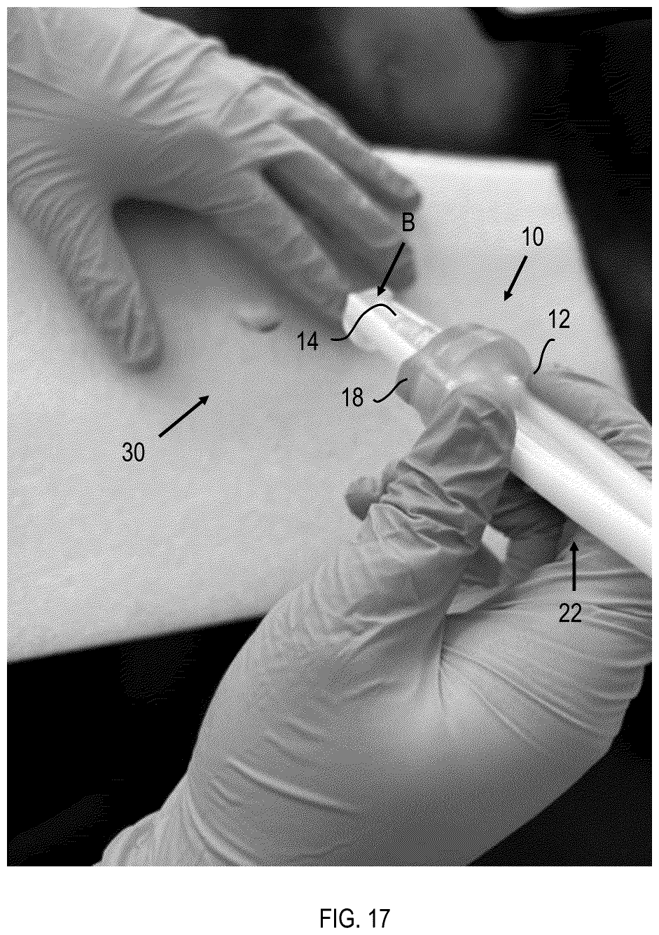

Once the cannula is so positioned (as detailed above) and the feeding mechanism 22 is removed, a user can twist or rotate the intermediate flange 18 down and toward the outer surface 30 (i.e., away from the second flange 12 as shown) along the corresponding threads on the cannulated body 16 such that the intermediate flange 18 is now positioned adjacent to the surface 30, stabilizing the cannula 10 (FIG. 11). When a user is ready to remove the cannula device 10 from the surface 30, the user can twist the intermediate flange 18 up and toward the second flange 12 (i.e., away from the surface 30), such that the flanges 12 and 18 are adjacent to each other toward the second end 10″ of the cannula 10 and the apertures thereof are aligned (FIG. 13). Next, a user can insert the arms 24 of the feeding mechanism 22 through the first apertures 12-1 of the second flange and elongated apertures 18-1 of the intermediate flange 18 and into and through the outer surface 30, the tissue, and the inner surface 30′ and use the arms 24 to pinch or squeeze the wings 14′ of the first flange 14, moving the wings 14′ to the engaged position B (FIGS. 14 and 15) (i.e., the outer perimeter portions of the first flange 14 move from facing opposite directions to facing substantially the same direction). Then, while still pinching the wings 14′ of the first flange 14, a user can pull the feeding mechanism 10 away from the surface 30′, tissue, and surface 30, thereby removing the first flange 12 and cannula 10 (FIG. 17) from the surgical site.

While embodiments of the present disclosure have been particularly shown and described with reference to certain exemplary embodiments, it will be understood by one skilled in the art that various changes in detail may be affected therein without departing from the spirit and scope of the disclosure as defined by claims that can be supported by the written description and drawings. Further, where exemplary embodiments are described with reference to a certain number of elements it will be understood that the exemplary embodiments can be practiced utilizing either less than or more than the certain number of elements.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as, “has” and “having”), “include” (and any form of include, such as “includes” and “including”), and “contain” (any form of contain, such as “contains” and “containing”) are open-ended linking verbs. As a result, a method or device that “comprises”, “has”, “includes” or “contains” one or more steps or elements. Likewise, a step of method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts and equivalents of all means or step plus function elements in the claims below, if any, are intended to include any structure, material or act for performing the function in combination with other claimed elements as specifically claimed. The description of the present disclosure has been presented for purposes of illustration and description, but is not intended to be exhaustive or limited to the embodiments in the form disclosed. Many modifications and variations will be apparent to those of ordinary skill in the art without departing from the scope and spirit of embodiments of the invention. The embodiments were chosen and described in order to best explain the principles of one or more aspects of the disclosure and the practical application, and to enable others of ordinary skill in the art to understand one or more aspects of the present invention for various embodiments with various modifications as are suited to the particular use contemplated.

What is claimed is:

1. A cannula, comprising:
a first flange positioned at a first end;
a second flange positioned at a second end; and
an elongated cannulated body extending along a central longitudinal axis and having a working portal extending therebetween;
wherein:
the first flange is flexible and is configured to move from a neutral position to a flexed position;
the first flange includes:
a first aperture, wherein the first aperture of the first flange is axially in line with the working portal; and
the second flange includes:
a first aperture, wherein the first aperture of the second flange is axially in line with the working portal and the first aperture of the first flange; and
a second aperture and a third aperture, wherein the second aperture and third aperture of the second flange are each adjacently positioned to the first aperture.

2. The cannula of claim 1, wherein the first flange further comprises a first outer perimeter portion and a second outer perimeter portion.

3. The cannula of claim 2, wherein the first outer perimeter portion and the second outer perimeter portion are oriented in opposing directions when in the neutral position.

4. The cannula of claim 3, wherein the first outer perimeter portion and the second outer perimeter portion are oriented in substantially the same direction when in the flexed position.

5. The cannula of claim 1, further comprising a third flange positioned along the cannulated body and between the first flange and the second flange.

6. The cannula of claim 5, wherein the third flange further comprises a central aperture and one or more elongated apertures formed therethrough.

7. The cannula of claim 6, wherein the third flange is moveable about the cannulated body.

8. The cannula of claim 7, wherein the cannulated body further comprises a plurality of threads integrally formed therewith that correspond to a plurality of threads integrally formed with the central aperture of the third flange to enable the third flange to rotate about the cannulated body and move closer to the first flange or the second flange.

9. The cannula of claim 8, wherein the elongated apertures of the third flange are configured to axially align with the second and third apertures of the second flange.

10. A cannula and inserter system, comprising:
a cannula comprising:
a first flange positioned at a first end;
a second flange positioned at a second end; and
an elongated cannulated body extending along a central longitudinal axis and having a working portal extending therebetween;
wherein:
the first flange is flexible and is configured to move from a neutral position to a flexed position;
the first flange includes:
a first aperture, wherein the first aperture of the first flange is axially in line with the working portal; and
the second flange includes:
a first aperture, wherein the first aperture of the second flange is axially in line with the working portal and the first aperture of the first flange; and
a second aperture and a third aperture, wherein the second aperture and third aperture of the second flange are each adjacently positioned to the first aperture; and
an inserter comprising:
a pair of elongated flexible arms having a first end and a second end and connected at the first end, wherein the elongated flexible arms are configured to be positioned through the second and third apertures of the second flange and a second end of each of the elongated flexible arms are configured to be positioned in contacting relation to the first flange and move the first flange from the neutral position to the flexed position.

11. The cannula and inserter system of claim 10, wherein the first flange further comprises a first outer perimeter portion and a second outer perimeter portion.

12. The cannula and inserter system of claim 11, wherein the first outer perimeter portion and the second outer perimeter portion are oriented in opposing directions when in the neutral position.

13. The cannula and inserter system of claim 12, wherein the first outer perimeter portion and the second outer perimeter portion are oriented in substantially the same direction when in the flexed position.

14. The cannula and inserter system of claim 10, further comprising a third flange positioned along the cannulated body and between the first flange and the second flange.

15. The cannula and inserter system of claim 14, wherein the third flange further comprises a central aperture and one or more elongated apertures formed therethrough.

16. The cannula and inserter system of claim 15, wherein the third flange is moveable about the cannulated body.

17. The cannula and inserter system of claim 16, wherein the cannulated body further comprises a plurality of threads integrally formed therewith that correspond to a plurality of threads integrally formed with the central aperture of the third flange to enable the third flange to rotate about the cannulated body and move closer to the first flange or the second flange.

18. The cannula and inserter system of claim 15, wherein the elongated apertures of the third flange substantially align with the second and third apertures of the second flange.

19. The cannula and inserter system of claim 15, wherein the inserter is configured to be positioned within the elongated apertures of the third flange and the second and third apertures of the second flange while in use.

20. The cannula and inserter system of claim 10, wherein the inserter further comprises a stopper provided on at least one of the elongated flexible arms and having a first end and a second end, wherein a length from the second end of the stopper to the second end of the elongated flexible arm is substantially equivalent to a length between the first and second ends of the cannula.
